(19) United States
(12) Patent Application Publication
(10) Pub. No.: US 2024/0115305 A1
(43) Pub. Date: Apr. 11, 2024

(54) LINEAR CRYOCATHETER

(71) Applicant: ArtFix LTD, Ariel (IL)

(72) Inventors: Lior LEVANONY, Bat Hefer (IL); Dvir KEREN, Tel Aviv (IL); Oron FELDMAN, Tel Aviv (IL)

(73) Assignee: ArtFix LTD, Ariel (IL)

(21) Appl. No.: 18/275,453

(22) PCT Filed: Feb. 2, 2022
(86) PCT No.: PCT/IL2022/050144

§ 371 Date: Aug. 2, 2023

Related U.S. Application Data
(60) Provisional application No. 63/144,512, filed on Feb. 2, 2021.

(51) Int. Cl. A61B 18/02

(52) U.S. Cl. CPC A61B 18/02; A61B 2018/00196; A61B 2018/0212; A61B 2018/0262

(57) ABSTRACT

Cryoablation device terminating in a linear ablation element, optionally with a slight curvature along its length. Upon advancing the linear ablation element along a longitudinal axis out of a catheter sheath, an angle selecting connector on the proximal end of the linear ablation element bends to re-orient the linear ablation element to extend radially outward from the longitudinal axis. The linear ablation element is then pressed to a lumenal tissue surface by advancing it along the longitudinal axis, rotating it around the longitudinal axis, and/or bending and/or rotating the sheath itself to reorient the longitudinal axis itself. Control degrees of freedom allow optionally placing the linear ablation element in a more distal position than it emerged from.

### RELATED APPLICATIONS

This application claims the benefit of priority under 35 USC § 119(e) of U.S. Provisional Patent Application No. 63/144,512 filed Feb. 2, 2021; the contents of which are incorporated herein by reference in their entirety.

### FIELD AND BACKGROUND OF THE INVENTION

The present invention, in some embodiments thereof, relates to the field of tissue ablation; and more particularly, but not exclusively, to cryoablation of tissue from within the lumenal space of an organ.

Currently, ablation is a gold standard therapy for patients who suffer from atrial fibrillation. While traditionally the ablation was done using RF means, an increasing segment of physicians uses a cryoballoon to achieve ablation. Similar to ablation by RF means, the cryoballoon catheter is inserted via an endovascular approach through the septum (i.e. trans-septally). The physician inflates the cryoballoon individually inside each of the four pulmonary veins, aiming to achieve an ablation in a ring-like geometry, along the connection of the pulmonary vein with the left atrium. The procedure may be repeated for each vein one or more times.

Ablation procedures may be re-performed in order to address gaps in ablations, including recovery from potentially incomplete ablations, and/or emergences of new and/or newly identified loci of electrical activity contributing to pathology.

### SUMMARY OF THE INVENTION

According to an aspect of some embodiments of the present disclosure, there is provided a cryoablation device configured to be inserted to a heart chamber via a transcutaneous, transvascular route, and including: a sheath, the sheath being actuatable to bend and thereby rotate a longitudinal axis of a distal end of the sheath to a selected angle within a range of angles between a straightened sheath angle, and at least 90° away from that angle; within the sheath, a cryogenic fluid conduit, actuatable to translate linearly along the longitudinal axis with respect to the sheath; a longitudinally extended ablation element: attached to a distal end of the cryogenic fluid conduit, sized to fit within the distal end of the sheath, and configured to receive coolant from the cryogenic fluid conduit to cool down to generate an longitudinally extended cryoablation surface along a longitudinal extent of the longitudinally extended ablation element; and an angle-selecting connector between the longitudinally extended ablation element and the fluid supply conduit, the angle-selecting connector bending to orient the longitudinally extended ablation surface to within about 20° of perpendicular to the longitudinal axis when the longitudinally extended element is outside the sheath.

According to some embodiments of the present disclosure, the longitudinally extended ablation surface extends at least 13 mm along the longitudinally extended ablation element.

According to some embodiments of the present disclosure, the longitudinally extended element curves less than 45° along a curvature following the at least 13 mm extent of the longitudinally extended ablation surface.

According to some embodiments of the present disclosure, the longitudinally extended ablation element includes a proximal side and a distal side, the proximal side is attached to the angled connection, and the distal side includes a terminus with a tip.

According to some embodiments of the present disclosure, the longitudinally extended element includes a tube of 1.8 mm diameter or less.

According to some embodiments of the present disclosure, the sheath holds the angle-selecting connector straightened while the longitudinally extended element is housed within the sheath, and the angle-selecting connector is configured to bend as the longitudinally extended element emerges from the sheath.

According to some embodiments of the present disclosure, the angle-selecting connector includes a metal piece with slits along it sides, and enclosed in flexible polymer tubing.

According to some embodiments of the present disclosure, the slits along the sides comprise gaps between coils of the angle-selecting connector.

According to some embodiments of the present disclosure, the slits along the sides comprise perforations.

According to some embodiments of the present disclosure, the perforations are positioned in opposite sets, to allow the metal piece to form a bend by contracting the perforations on an inner side of the bend, and expanding the perforations on an outer side of the bend.

According to some embodiments of the present disclosure, the angle-selecting connector is short enough to allow the sheath distal end to remain within about 1 cm of the cryoablation surface of the longitudinally extended ablation element when deployed to contact and ablate tissue.

According to some embodiments of the present disclosure, the angle-selecting connector is short enough that a proximal end of the longitudinally extended ablation element remains within about 1 cm of longitudinal axis when both it and the angle-selecting connector are emerged from within the sheath.

According to some embodiments of the present disclosure, the angle-selecting connector bends through a radius of curvature in a range of about 3-10 mm.

According to some embodiments of the present disclosure, once the angle-selecting connector and the longitudinally extended ablation element are emerged from the sheath, the cryogenic fluid conduit is configured to allow further actuated advance along the longitudinal axis, while the sheath distal end remains in place.

According to some embodiments of the present disclosure, the cryogenic fluid conduit is configured to rotate within the sheath, thereby selecting an angular position of the longitudinally extended ablation element around the longitudinal axis when emerged from the sheath.

According to some embodiments of the present disclosure, the distal end of the sheath is bendable to assume at least 135° of curvature, while the longitudinally extended ablation element is emerged from the sheath.

According to some embodiments of the present disclosure, bending of the sheath is controllable up to an angle of at least 155°.

According to some embodiments of the present disclosure, bending of the sheath is through a radius of curvature of less than about 5 cm.

According to some embodiments of the present disclosure, the longitudinally extended ablation element includes a superelastic alloy which softens at the cryoablation temperature to allow redistribution of contact forces between the distal end of the longitudinally extended ablation element and the proximal side of the longitudinally extended ablation element.

According to some embodiments of the present disclosure, the cryoablation device includes at least one thermocouple device positioned between the cryogenic fluid conduit and a distal tip attached to the distal end of the longitudinally extended ablation element.

According to some embodiments of the present disclosure, the cryoablation device includes at least one electrode attached to the longitudinally extended ablation element, and configured to sense electrophysiological signals from tissue while the ablation surface is pressed into contact with tissue targeted for ablation.

According to some embodiments of the present disclosure, the longitudinally extended ablation element includes a tubular element, and the coolant circulates bidirectionally through at least the proximal end of the tubular element.

According to some embodiments of the present disclosure, the cryoablation device includes a handle with an actuation control for linear translation of the cryogenic fluid conduit relative to the sheath.

According to some embodiments of the present disclosure, the handle includes an actuation control for rotating the cryogenic fluid conduit relative to the sheath.

According to some embodiments of the present disclosure, the handle includes an actuation control for bending the sheath.

According to some embodiments of the present disclosure, the longitudinally extended ablation element includes: a first curvature distal to curvature of the angle-selecting connector; and a second curvature distal to the first curvature, and in an opposite direction; wherein the first curvature is oriented to fit against an interior surface portion of a body tissue lumen, and the second curvature is oriented to elevate a tip of the longitudinally extended ablation element away from tissue adjacent to the interior surface portion.

According to some embodiments of the present disclosure, the first curvature is sized and shaped to fit against a cavo-tricuspid isthmus, and the second curvature elevates the tip away from contact with leaflets of a tricuspid valve adjacent the cavo-tricuspid isthmus when the first curvature is placed against the cavo-tricuspid isthmus.

According to an aspect of some embodiments of the present disclosure, there is provided a method of operating a cryoablation device including: inserting a sheath of the device through an entry aperture in a tissue wall; bending a portion of the sheath inserted past the tissue wall to an angle of more than 90°; linearly translating a cryofluid conduit along a longitudinal axis of a distal portion of the sheath, causing a longitudinally extended ablation element to emerge from the sheath and rotate to assume an orientation within about 20° of a plane perpendicular to the longitudinal axis; rotate the longitudinally extended ablation element around the longitudinal axis by rotation of the cryofluid conduit; and further linearly translating the cryofluid conduit to bring a cryoablation surface of the longitudinally extended ablation element into contact with tissue of the tissue wall, within 2 cm of the entry aperture.

According to some embodiments of the present disclosure, the method includes partially withdrawing the inserted portion of the sheath to bring the cryoablation surface into contact with tissue of the tissue wall.

According to some embodiments of the present disclosure, the method includes bending the portion of the sheath is through an angle of more than 135°.

According to an aspect of some embodiments of the present disclosure, there is provided a method of operating a cryoablation device including: placing a ring-shaped portion of an electrode catheter at the ostium of a pulmonary vein; placing a longitudinally extended cryoablation element in contact with tissue alongside the ring, and oriented approximately parallel to a tangent to a nearest portion of the ring; ablating, using the longitudinally extended cryoablation element; and repeating the placing in a tangential orientation and ablating adjacent to the ring an additional plurality of different nearest portions of the ring.

According to an aspect of some embodiments of the present disclosure, there is provided a cryoablation device configured to be inserted to a heart chamber via a transcutaneous, transvascular route, and including: a longitudinally extended ablation element including an ablation surface extending between a proximal end and a distal end of the longitudinally extended ablation element, and configured to be cooled, while in contact with tissue, to a cryoablation temperature by the circulation of coolant through the longitudinally extended ablation element; a sheath containing the longitudinally extended ablation element, and defining a longitudinal axis extending along a distal end of the sheath and past a distal opening of the sheath; a cryogenic fluid conduit configured to deliver the coolant to the longitudinally extended ablation element; and an angle-selecting connector, interconnecting a distal end of the cryogenic fluid conduit to the proximal end of the longitudinally extended ablation element; wherein the angle-selecting connector is configured to bend, as it emerges from a distal end of the sheath, from a straightened configuration extending along the longitudinal axis to an angled configuration, thereby re-orienting the longitudinally extended ablation element to a deployed angle of at least 70° relative to the longitudinal axis; and wherein the ablation surface, in the re-oriented position of the longitudinally extended ablation element: is oriented to contact tissue on a side of the longitudinally extended ablation element facing away from the sheath, has a proximal end within 1 cm of the longitudinal axis, and curves less than 45° around a radius of a curvature extending between the proximal side and the distal side of the longitudinally extended ablation element.

According to some embodiments of the present disclosure, the radius of curvature is greater than 10 cm.

According to some embodiments of the present disclosure, the angle-selecting connector is short enough to allow the sheath to remain within about 1 cm of the longitudinally extended ablation element while it contacts tissue.

According to some embodiments of the present disclosure, the distal side of the longitudinally extended ablation element is within 2 cm of the longitudinal axis.

According to some embodiments of the present disclosure, the distal side of the longitudinally extended ablation element is at least 13 mm away from the proximal side of the ablation surface.

According to some embodiments of the present disclosure, the cryogenic fluid conduit rotates with respect to the sheath, thereby inducing rotation of the longitudinally extended ablation element deployed at the deployed angle around the longitudinal axis.

According to some embodiments of the present disclosure, the distal end of the sheath is actuatable to assume at least 135° of curvature, while the longitudinally extended ablation element is deployed at the deployed angle.

According to some embodiments of the present disclosure, the longitudinally extended ablation element deployed at the deployed angle is sized to be reoriented, while the distal end of the sheath is curved, so that a distal end of the longitudinally extended ablation element reaches to within 2 cm of the sheath.

According to some embodiments of the present disclosure, the angle-selecting connector includes a metal piece with slits along it sides, and enclosed in flexible polymer tubing.

According to some embodiments of the present disclosure, the slit sides are constructed of coiled metal, and the slits are gaps between the coils.

According to some embodiments of the present disclosure, the slit sides are constructed of perforated metal.

According to some embodiments of the present disclosure, the perforations are positioned to allow the sides of the metal piece to form a bend by contracting the perforations on an inner side of the bend, and expanding the perforations on an outer side of the bend.

According to some embodiments of the present disclosure, the cryoablation device includes a handle with an actuatable element for rotating the cryogenic fluid conduit relative to the sheath.

According to some embodiments of the present disclosure, the cryoablation device includes a handle with an actuatable element for linearly translating of the cryogenic fluid conduit relative to the sheath.

According to some embodiments of the present disclosure, the cryoablation device includes a handle with an actuatable element for inducing controlled bending of the sheath.

According to some embodiments of the present disclosure, the controlled bending is up to an angle of at least 155°.

According to some embodiments of the present disclosure, the controlled bending is through a radius of curvature of less than about 5 cm.

According to some embodiments of the present disclosure, the angle selecting connector bends to re-orient the longitudinally extended ablation element by bending through a radius of curvature less than about 2 cm.

According to some embodiments of the present disclosure, the longitudinally extended ablation element includes a superelastic alloy which softens at the cryoablation temperature to allow redistribution of contact forces between the distal end of the longitudinally extended ablation element and the proximal side of the longitudinally extended ablation element.

According to some embodiments of the present disclosure, the cryoablation device includes at least one thermocouple device positioned between the cryogenic fluid conduit and a distal tip attached to the distal end of the longitudinally extended ablation element.

According to some embodiments of the present disclosure, the cryoablation device includes at least one electrode attached to the longitudinally extended ablation element, and configured to sense electrophysiological signals from tissue while the ablation surface is pressed into contact with tissue targeted for ablation.

According to some embodiments of the present disclosure, the longitudinally extended ablation element includes a tubular element, and the coolant circulates bidirectionally through at least the proximal end of the tubular element.

According to an aspect of some embodiments of the present disclosure, there is provided a method of operating a cryoablation device, the method including: advancing a longitudinally extended ablation element out of a distal aperture of a sheath of the cryoablation device; bending the sheath and reorienting the longitudinally extended ablation element so that a distal tip of the longitudinally extended ablation element reaches to within 2 cm of the sheath at a position which is closest to the sheath at a location along the sheath which is proximal to the distal aperture; and placing the longitudinally extended ablation element in contact with tissue and cooling it to ablate the tissue.

According to an aspect of some embodiments of the present disclosure, there is provided a method of operating a cryoablation device, the method including: extending a longitudinally extended ablation element out of a sheath of the cryoablation device; re-orienting the longitudinally extended ablation element from a packaged orientation extending along the longitudinal axis to a deployed orientation of at least 70° relative to the longitudinal axis, while a proximal end of the longitudinally extended ablation element remains within about 10 mm of the sheath; placing the longitudinally extended ablation element into contact with tissue along an ablation surface extending between a distal end and the proximal end of the longitudinally extended ablation element, through a distance of at least 13 mm; and ablating the tissue by cooling the longitudinally extended ablation element; wherein the ablation surface faces away from the sheath, and a curvature of the ablation surface.

Unless otherwise defined, all technical and/or scientific terms used herein have the same meaning as commonly understood by one of ordinary skill in the art to which the present disclosure pertains. Although methods and materials similar or equivalent to those described herein can be used in the practice or testing of embodiments of the present disclosure, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the present disclosure are herein described, by way of example only, with reference to the accompanying drawings. With specific reference now to the drawings in detail, it is stressed that the particulars shown are by way of example, and for purposes of illustrative discussion of embodiments of the present disclosure. In this regard, the description taken with the drawings makes apparent to those skilled in the art how embodiments of the present disclosure may be practiced.

In the drawings:

FIG. 1A schematically represents a distal section of a cryoablation device, according to some embodiments of the present disclosure.

FIG. 1B schematically represents a cryoablation device, including control elements, according to some embodiments of the present disclosure;

FIG. 1C schematically illustrates aspects of lumenal surface accessibility provided by the degrees of control freedom of a cryoablation device, according to some embodiments of the present disclosure;

FIGS. 1D-1E show a distal section of a cryoablation device in cross-section, according to some embodiments of the present disclosure;

FIG. 2A schematically represents a sequence of configurations assumed by cryoprobe during its advance from sheath, according to some embodiments of the present disclosure;

FIGS. 2B-2C schematically represent rotational control of cryoprobe, according to some embodiments of the present disclosure;

FIG. 3 schematically represents a sequence of configurations illustrating flexing control of catheter sheath of a cryoablation device, according to some embodiments of the present disclosure;

FIG. 4 schematically represents a sequence of configurations illustrating translational control of cryoprobe with respect to sheath, according to some embodiments of the present disclosure;

FIG. 5 is a flowchart schematically representing a method of operation of cryoablation device, according to some embodiments of the present disclosure;

FIGS. 6A-6B schematically illustrate positioning of cryoablation device within a left atrium of a heart, according to some embodiments of the present disclosure;

FIG. 7 schematically illustrate positioning of cryoablation device within a left atrium of a heart, according to some embodiments of the present disclosure;

FIGS. 8A-8F schematically and with angiographic images represents positioning of cryoablation device within a left atrium, according to some embodiments of the present disclosure;

FIGS. 9A-9B schematically illustrate components of an assembly comprising angle-selecting connector and linear ablation element, according to some embodiments of the present disclosure;

FIG. 10A-10D schematically illustrate use of linear ablation elements, shaped to conform to the shape of a targeted ablation region, while avoiding tissue of adjacent non-targeted tissue, according to some embodiments of the present disclosure; and

FIG. 10E schematically illustrates expanded views of linear ablation elements, according to some embodiments of the present disclosure.

### DESCRIPTION OF SPECIFIC EMBODIMENTS OF THE INVENTION

The present invention, in some embodiments thereof, relates to the field of tissue ablation; and more particularly, but not exclusively, to cryoablation of tissue from within the lumenal space of an organ.

### Overview

An aspect of some embodiments of the present disclosure relates to devices for performing cryogenic ablations within body lumens using a linearly extended ablation element. In some embodiments, the body lumen is a heart chamber, for example, a left atrium of a human heart. In some embodiments, access to the body lumen is via a restricted passageway, e.g., a transseptal crossing location between a right atrium and a left atrium. In some embodiments, access to the heart is via the percutaneous and optionally transvascular introduction of an ablation treatment device. In the heart cryogenic ablation (cryoablation) is a treatment performed to modify heart electrical activity, e.g., as a method of disrupting transmission pathways which play a role in atrial fibrillation.

International Patent Publication No. WO2002/240548, the contents of which are incorporated herein by reference in their entirety, describes ablation devices configured for circulation of cryogenic fluid to and through the distal end of an ablation catheter. Some embodiments of the present disclosure operate according to one or more of the mechanisms and/or principles described therein to circulate coolant, and achieve thereby cooling capable of ablating heart tissue. In some embodiments, the coolant circulates bidirectionally through a single tubular element, that is, it flow both in and out of a same proximal side of the tubular element, between which is circulates within the tubular element.

In the course of their investigations, the inventors of the present application have found that there are potential cryoablation applications for which contact surfaces having an extent of about ⅓ to ½ the trans-chamber length of the atrium may be well-suited. In particular, although not exclusively, heart tissue ablations under the current standard of care often are performed in a series of two or more separate procedures. A first ablation procedure may be followed, immediately or after a period of a few months or more, by recurrence of fibrillation due to incomplete interruption of the pathological electrical pathways, recovery of incompletely ablated tissue, and/or re-routing. Often, this is attributed to the remainder and/or development of a “gap” along the ablation line which the first procedure tried to establish. In some cases, a follow-up procedure is performed which seeks to identify and eliminate such gaps.

For this purpose, a tool which ablates along a moderately large extent along the heart's inner surface may provide an advantage over point ablation devices, for example by reducing the precision with which the gap location needs to be identified, and/or by reducing the precision with which the ablation tool needs to be positioned in order to address it. It is also a potential advantage to be able to ablate longer extents with a single operation, as this can reduce procedure time; and/or free more procedure time for exploratory measurements and/or decision making. Nevertheless, when an ablation has already been performed, the gaps are generally expected to be localized, so ablating, e.g., along a surface extending the whole width or length of a heart chamber is less desirable; and may gain nothing useful compared to an ablation surface which extends (for example) for about one quarter, one third, or one half of the length of the heart chamber.

An aspect of some embodiments of the present disclosure relates to the control of position of devices for performing cryogenic ablations within body lumens using a linearly extended ablation element.

Another potential advantage of providing a linearly extended contact surface for ablation is that it can assist in reaching locations which may otherwise be difficult to treat. There is in general a lower limit to the radius of curvature allowed by a steerable catheter end, e.g., a limit imposed by the compressibility of the curve-interior side of the catheter end, and/or the extensibility of the curve-exterior side of the catheter end. Accordingly, a 180° bend doesn't point the catheter tip back at it itself (i.e., back at its own entryway into the heart chamber), but instead some way off, e.g., 3-5 cm, or another value according to the radius of curvature achieved. For tools which exit the catheter at a straight angle (tangent to the final curve of the catheter end), this creates a potentially inaccessible zone of the same radius. The curvature may (potentially with difficulty) be extended past 180°, but then the final tangent line is also bent, which potentially interferes with the ability to establish and maintain a firm contact with a targeted location without sliding out of position, or otherwise creating targeting uncertainty.

The ability of a probe to solve the “back-access problem” just described may be taken as indicative more generally of the flexibility and maneuverability of the probe, available to whatever other position (behind, to the side, or in front) the probe needs to perform its unction. Even if there is no particular need, e.g., to ablate back onto the septal wall, having sufficient flexibility to do so may be advantageous in operation of the device more generally. Even if an area can be reached, it is not necessarily possible to treat it effectively from every angle. For example, an approximately perpendicular approach to a tissue target may be needed to achieve a stable positioning of a probe before ablation begins. A physician may find it easier to track progress of a procedure if positioning can be performed by a regular, reliable pattern, and greater maneuverability options may potentially help the physician find the pattern that suits their particular working preferences.

In some embodiments of the present invention, the back-access problem, and/or the problem of maneuverability more generally, is addressed by a number of features, optionally provided separately or in combination.

The first feature is the linearly extended ablation contact surface of the cryoablation probe itself. In this connection, the term “linear” should be understood to indicate that the surface has a distal end and a proximal end, with the shortest travel distance along the probe between the two ends being, for example, at least 10 mm, 12 mm, 13 mm, 15 mm, 18 mm, 2 cm, 2.5 cm, or another distance. The linear (linearly extended) ablation contact surface is not necessarily straight, however, and in some embodiments is curved, e.g., to enhance fitting against tissue of a lumenal surface. Furthermore, in some embodiments, approximately perpendicular to the shortest travel distance, the probe has a largest diameter which is significantly smaller than the shortest travel distance, e.g., about 2 mm, 1.5 mm 1 mm, or another diameter; for example, a diameter in the range of about 0.8 mm to 1.8 mm. In some embodiments, the probe has a circumference approximately perpendicular to the shortest travel distance which is about 6 mm 4.5 mm, 3 mm, or another circumference.

The proximal and distal ends of the linearly extended ablation contact surface are distinguished, in some embodiments, by their respective relationships to the geometry of a longitudinally extended ablation element which defines the contact surface. The longitudinally extended ablation element, in some embodiments, comprises a connected end and a tipped terminus. The longitudinally extended ablation element is optionally curved, while still circulating cryofluid back to the cryofluid conduit without looping back on itself to do so. The proximal end of the contact surface is the end closer to a proximal end of a longitudinally extended ablation element through which connection is made for the delivery of cryofluid into the longitudinally extended ablation element. The distal end of the contact surface is the end closer to a distal end of a longitudinally extended ablation element which is a terminus, the terminus bearing, e.g., a distal tip. Circulation of cryofluid within the longitudinally extended ablation element is bidirectional, i.e., both introduced and vented through the same proximal end.

Optionally, longitudinally extended ablation element has no free terminus, and is connected back to the cryofluid conduit on both its ends to form a loop. This allows, e.g., unidirectional circulation of coolant through the loop. Curvatures and/or relative lengths of the members which connect the longitudinally extended ablation element back to the cryofluid conduit determine its deployed position and orientation relative to the distal end of the cryofluid conduit. For example, it may be positioned, when deployed, centered on a longitudinal axis of the distal end of the cryofluid conduit and perpendicular to it; or, optionally, obliquely and/or off-center. Leaving one end free has some potential advantages, however. With respect to pre-deployment packaging of the device, For example, the packaged longitudinally extended ablation element with a free distal terminus does not require sharp bending or doubling over on itself through a flexible region, which may potentially create a point of mechanical failure. The free distal terminus also allows a potentially larger disk of “reach” (for a given linear size of its contact surface) to be accessed from any given position of the distal terminus of the cryofluid conduit. It may be difficult to arrange a double-end connected longitudinally extended ablation element which deploys to a useful shape even when only partially extended, while the angulation of singly-connected linear ablation element is optionally varied by partially extending it to a greater or lesser extent. A fully deployed single-end connected longitudinally extended ablation element may be more compact than a double-end connected ablation element of the same longitudinal extent, e.g., it may be usable within a smaller space, at a shorter axial distance from a sheath aperture from which it deploys. It should be understood that single-end connected longitudinally extended ablation elements are optionally replaced with double-end connected ablation elements, recognizing that this may modify or abolish certain features such as a clear proximal-distal axis of the element, and/or its freedoms and/or modes of movement.

The contact surface may be straight, or optionally curved along the shortest proximal-distal travel distance. Preferably, the curvature is relatively gentle; e.g., corresponding to a radius of curvature in the range of 15-45 mm or more; for example, 15 mm, 30 mm, or 45 mm, on an outer diameter of about 1-1.8 mm. The radius of curvature may be set to a value which is about equal to and preferably larger than the radius of the heart chamber. Thus, in some embodiments, the change along the travel distance in angular direction is, for example, about 30° or less, 45° or less, or 15° or less. It is noted that the ablation surface is preferably on the convex side of the curvature. There may be some flexibility in the cryoablation probe, allowing it to flex (particularly as it cools) to potentially better conform to tissue against which it is pressed.

For the length of the linearly extended ablation surface to be used effectively, it should be pressed against tissue along substantially its whole length. During delivery of the device, it first travels along longitudinal (proximal-to-distal) axis of a sheath that contains it, with its own proximal-to-distal axis oriented about parallel to that of the sheath it travels in. Upon existing the sheath, however, the cryoablation probe re-oriented so that it extends roughly at a right angle to its pre-emergence orientation (e.g., it re-orients by about 70° or more). This is the second feature which assists in addressing the back-access problem. It is in this orientation that the ablation surface makes contact with tissue against which it is force. In effect the “sliding off target” problem of a tangent exiting probe is mitigated by bending the cryoablation probe so much that it's already bent when it makes contact. And if not yet all the way bent, the additional bending it does may serve to increase tissue-probe contact, rather than to skidding off-target.

In some embodiments, the re-orientation is achieved using a superelastic member (e.g., constructed of nitinol), which is preset to adopt an angle of about 70° or more when unconstrained (e.g., when it exits its sheath). In some embodiments, the angle is not under external control while the cryoprobe is fully extended, although it may exhibit some compliance when the cryoprobe is forced against tissue, so that the actual angle it assumes may vary from its preset value by several degrees, e.g., up to 10° more, 15° more or 20° more. Under visualization (e.g., angiographic visualization) this compliance potentially provides a visual cue indicative of contact quality between cryoprobe and tissue.

A third feature, in some embodiments, is that the cryoprobe can be rotated separately from its sheath, e.g., rotated enough to sweep through a full 360°, and optionally up to several turns. Less than 360° may also be sufficient. The rotation may be driven, e.g., by a hand-held controller positioned outside the body, which delivers torque along the length of the device from proximal to distal until it causes the cryoprobe itself to turn.

Combining the first two features with this third feature, in some embodiments, the cryoprobe is manipulatable to sweep out an approximately disk-shaped (or highly-flattened cone-shaped) surface. Furthermore, a 180° bend in the catheter sheath, coupled to an appropriate rotation of the cryoprobe, can bring a distal tip of the ablation surface back toward the radial center along which the sheath's longitudinal axis extends. Optionally, the cryoprobe tip reaches the sheath, or reaches to within 5 mm, 10 mm, 15 mm, or 20 mm of the sheath, even if the radius of curvature of the sheath itself is greater than 15 mm, 20 mm, 30 mm, or more. How close it can reach depends in part on the relationship of the length of the cryoprobe to the radius of curvature of the sheath. For example, if the cryoprobe is near to twice the length of the radius of curvature, it may be long enough that the sheath interferes with rotation of the cryoprobe when it is rotated while the sheath is fully bent backward.

In some embodiments, an additional degree of freedom is obtained by adjusting the relative advance of the cryoprobe past the distal end of the steerable sheath. There are further at least partial degrees of freedom, insofar as the steerable sheath itself can be advanced or rotated. These different degrees of freedom may be maneuvered too adjust the angle of approach made with tissue, for example, to select a nearly perpendicular angle of approach, or another angle.

It should also be noted that these degrees of freedom may be manipulated so that many regions of the lumenal surface can be crossed from substantially any direction. Just as there is a “disk” (or other rotationally symmetric shape) of ablation orientations available from each central contact point of the probe (absent interfering structures within the lumen), there is a complementary disk of contact points from which each surface location on the body lumen interior can be reached by some part of the cryoablation probe.

This potentially allows considerable flexibility in planning ablations around the anatomical and physiological particulars of any given ablation treatment situation. For example, a gap may be determined (e.g., based on electrophysiological measurements, performed by a separate device and/or in some embodiments by electrophysiology electrodes on the cryoablation device itself) to have a longitudinal axis lying between two well-ablated regions; the probe is optionally oriented to be parallel to that axis when it is operated to ablate. Moreover, if the gap is in a curved ablation line, the cryoablation probe can be moved to the interior of the curve, so that it is more likely to intersect it in two already well-ablated positions, closing the gap. Optionally, the probe is oriented to thread between anatomical features, e.g., midway between two pulmonary arteries. Alternatively, the effective length of the ablation can optionally be shortened, in some instances, by deliberately positioning a portion of the ablating surface over a gap created by a nearby feature such as a pulmonary vein. This may be of use when the correction needed is judged to be small, e.g., to avoid overproduction of new scarring in an already ablated heart chamber.

In another example, some areas may be determined to lie within a fissure with a particular orientation. The cryoprobe is optionally oriented to lie along the fissure, so that it can slip into it, rather than be held suspended across it.

In another example, some areas may be difficult from all angles, at least as far as access to a linear-shaped ablation surface is concerned. In such cases, it may be preferable to partially retract the cryoprobe into its sheath until it again assumes a more longitudinal orientation with respect to the sheath. There may still be a slight amount of rotational freedom of movement, if the probe is off-angle, or if this is not useful or usable, the device operator still has other degrees of control freedom which may allow access to ablate in a more punctate ablation form, using the probe tip and/or distal side of the probe tip.

It should be understood that the same device is optionally used to perform some or all of the ablations in a first heart ablation. The linear shape of the catheter can be used to isolate tissue regions, e.g., with polygonal shapes and/or chained linear sequences of ablations.

An aspect of some embodiments of the present disclosure relates to longitudinally extended ablation elements shaped to fit against particular preselected surface features within a body lumen having a distinctive shape.

In some embodiments, a longitudinally extended ablation element is shaped to extend into a hooked shape comprising at least two opposite directions of curvature. The concave side of a first of the curvatures defines a linearly extended ablation contact surface, shaped and sized to hook over an interior surface of the body lumen. A second of the curvatures lifts a section of the longitudinally extended ablation element more distal to the first curvature away from the interior surface of the body lumen.

A potential advantage of the second curvature is to maintain the distal section away from tissue which is not targeted for ablation. The option of simply truncating the distal section before non-targeted tissue is reached may be undesirable and/or difficult to implement. For example, this may make the distal end too sharp—if pulled too hard, it may puncture tissue. In some embodiments, a distal end of the longitudinally extended ablation element comprises an enlargement at the tip. The tip can itself be cooled by when coolant is released into the longitudinally extended ablation element, but is optionally not intended as part of the ablation surface (i.e., contact with the cooled tip may be potentially damaging, yet it may not be reliably useful for ablating with). Making the longitudinally extended ablation element shorter may tend to bring the tip into interference with the intended ablation surface. The second curvature allows maintaining a target length of the ablation surface while also keeping the tip away from body lumen surfaces.

Before explaining at least one embodiment of the present disclosure in detail, it is to be understood that the present disclosure is not necessarily limited in its application to the details of construction and the arrangement of the components and/or methods set forth in the following description and/or illustrated in the drawings. Features described in the current disclosure, including features of the invention, are capable of other embodiments or of being practiced or carried out in various ways.

### Cryocatheter Elements

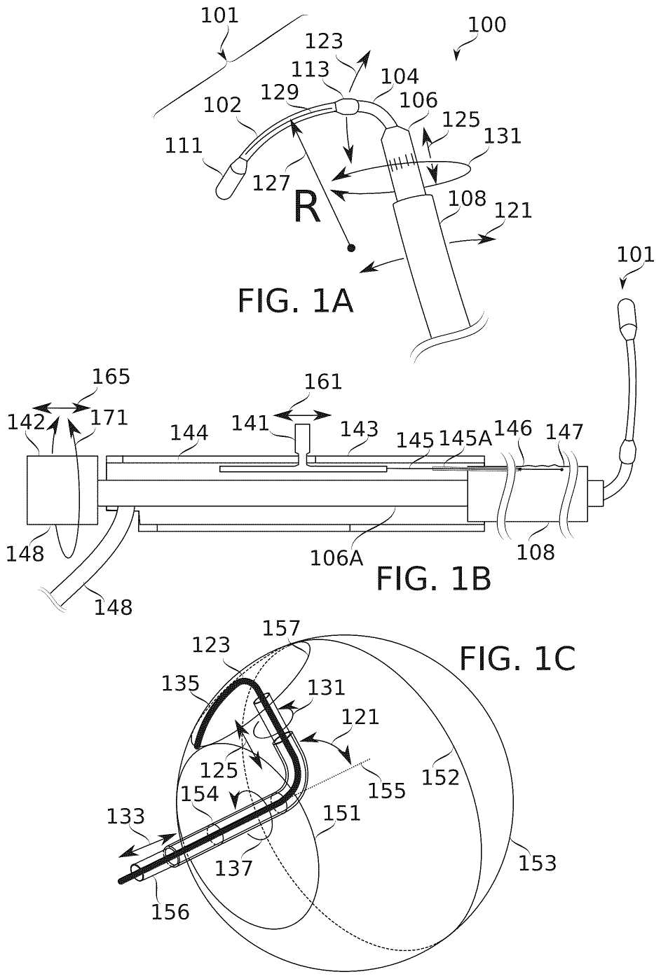

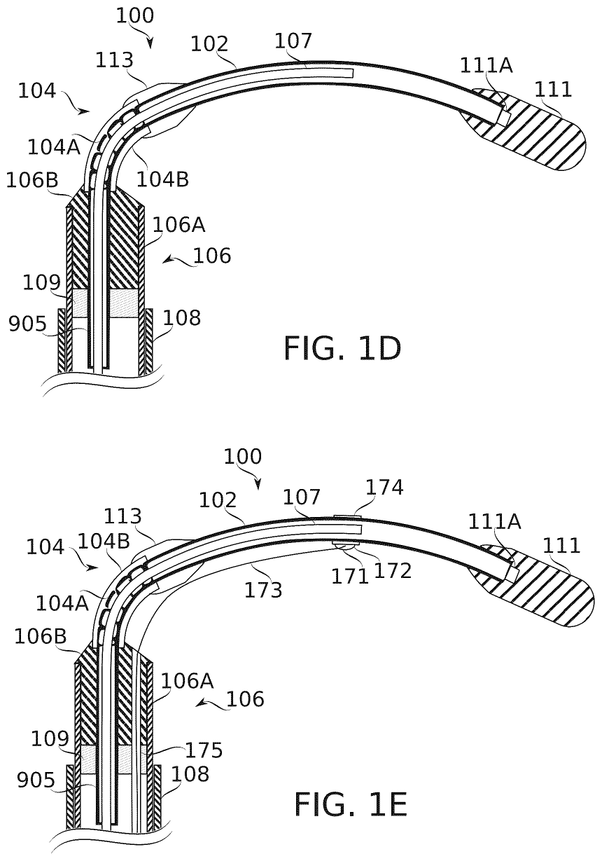

Reference is now made to FIG. 1A, which schematically represents a distal section of a cryoablation device 100, according to some embodiments of the present disclosure. Reference is also made to FIGS. 1D-1E, which show a distal section of a cryoablation device 100 in cross-section. In FIG. 1E, placement and connections of a thermocouple device 171 are indicated.

In some embodiments, cryoablation device 100 comprises cryoablation probe 101, angle-selecting connector 104, cryogenic fluid conduit 106, and steerable sheath 108. A typical outer diameter for steerable sheath 108 is about 3-6 mm, with the sheath being steerable with a bending radius down to a radius of curvature of about 8-25 mm. Cryogenic fluid conduit 106 may have an outer diameter of about 2.5-5 mm, and an inner diameter of about 2-4 mm.

Cryoablation probe 101, in some embodiments, comprises longitudinally extended ablation element 102, tipped on a distal end by tip 111, and attached on a proximal side via seal 113 to angle-selecting connector 104. In some embodiments, tip 111 comprises a hollow 111A in communication with the lumen of longitudinally extended ablation element 102, which potentially assists in cooling of tip 111 (for example cooling sufficient to allow tip 111 to reach temperatures which freeze it to target tissue).

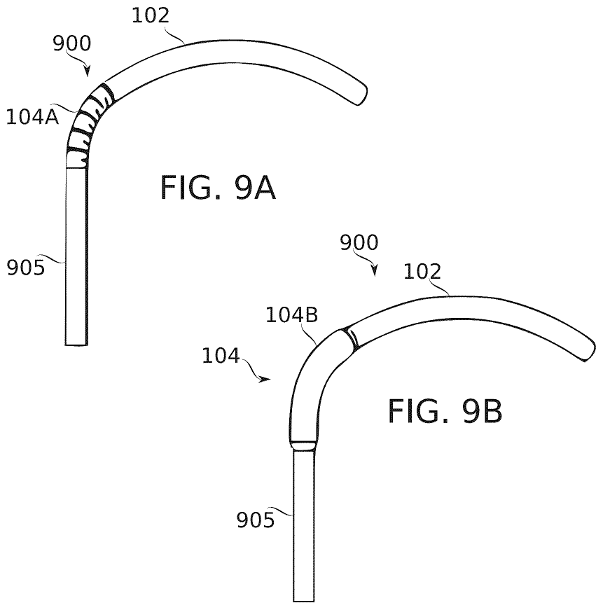

Brief reference is made to FIGS. 9A-9B, which schematically illustrate components of an assembly comprising angle-selecting connector 104 and longitudinally extended ablation element 102. In some embodiments, a single piece of metal tubing 900 (e.g., nitinol tubing) is formed with the following three sections:
proximal end 905, which provides length that can be sealed around;
slit section 104A, which is slit (e.g., laser-cut) to allow flexibility of the bending of angle-selecting connector 104; and
longitudinally extended ablation element 102, which may be straight or curved as shown.

In some embodiments, a radius of curvature through the angle-selecting connector 104 is in a range of about 3-10 mm. A radius of curvature 127 through the longitudinally extended ablation element 102 (e.g., along curve segment 129) is about 15-45 mm, in some embodiments. Optionally the tube which comprises these curvatures has an outer diameter of about 0.8-1.8 mm, and an inner diameter of about 0.6-1.3 mm.

Tubing 104B (FIG. 9B), over slit section 104A, closes off the slits. Tubing 104B may comprise a heat-shrink polymer such as a polyurethane or PTFE, and/or PET, polyacrylamide, and/or polyester. Additional sealing is optionally provided at the ends using an epoxy and/or cyanoacetate glue. For example, optionally additional sealing is provided at seal 113 (FIGS. 1A, 1D-1E), and within cryogenic fluid conduit 106, which comprises, e.g., plug 106B, fitted within tube 106A, which may itself be thermally insulated. In some embodiments, a plate 109 is provided which helps to stabilize the positioning of elements which connect at and/or pass through a distal end of cryogenic fluid conduit 106.

Slitting of the metal tube portion of angle-selecting connector 104 (as well as the provision of polymer overtube sealing) is optional, but the laser-cut slitting potentially allows a bend with less resistance to deformation and/or a smaller radius of curvature to be constructed.

In operation to ablate, cooling fluid (e.g., liquefied and/or pressurized N₂O, N₂, and/or a nitrogen-oxygen mixture), is supplied to the distal section of cryoablation device 100 via cryogenic fluid conduit 106. In some embodiments, cryogenic fluid conduit 106 houses supply conduit 107, which may pass into proximal end 905 of metal tubing 900, and terminate distally at one or more apertures located within longitudinally extended ablation element 102. Cooling may be assisted by evaporative and/or adiabatic cooling, upon a drop in pressure upon exiting supply conduit 107, for example, a drop from about 30-40 bar to a pressure several times lower, e.g., about 1 bar.

Cryofluid passing out of this distal termination cools (absorbs heat energy from) longitudinally extended ablation element 102, chilling it to temperatures sufficiently low to induce ablation of tissue by contact therewith; for example, a temperature of −40° C. down to about −80° C. or below. The fluid then passes back out of proximal end 905, e.g., through a gap between an inner lumenal wall of longitudinally extended ablation element 102, and the outer wall of supply conduit 107. The spent coolant then recirculates out of the body by passing back through the lumen of cryogenic fluid supply conduit 106.

In some embodiments, fluid eventually reaches tip 111, wherefrom it is redirected to recirculate back through ablation element 102, join 113, connector 104, and cryogenic fluid conduit 106. The redirection, in some embodiments, comprises direction of cryogenic fluid into a lumen separate from the series of lumens which supplied the cryogenic fluid. Examples of internal details of devices which receive cryogenic fluid, use it to chill an outer surface of an ablation probe, and then return the fluid are described, for example, in International Patent Publication No. WO2002/240548, the contents of which are incorporated herein in their entirety.

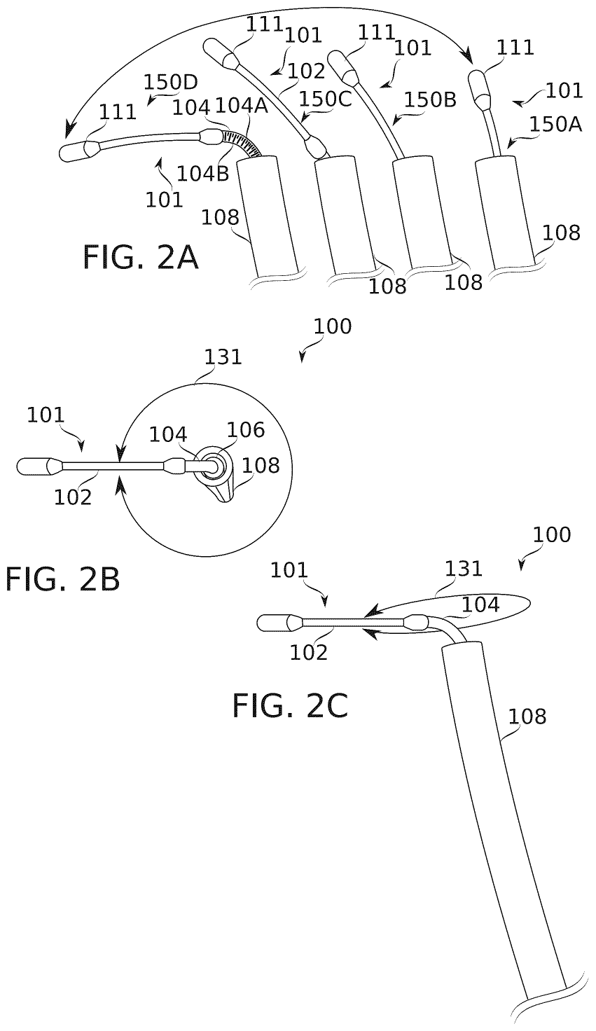

In some embodiments, angle-selecting connector 104 is a passively deformable section of cryoablation device 100. Connector 104 adopts a bend approaching 90° or more when unconstrained (e.g., at least a 70° bend). While constrained within sheath 108, connector 104 is forced into a straighter configuration, so that it and cryoablation probe 101 are oriented longitudinally along a lumen of sheath 108. Angle change during emergence of cryoablation probe 101 from sheath 108 is described, for example, in relation to FIG. 2A.

Actuatable movements of cryoablation 100 include induced curvature 121 of sheath 108 (mono- or bi-directional), rotation 131 relative to sheath 108 of cryogenic fluid conduit 106 of up to 360° or more (by this rotation, cryoablation probe 101 is also rotated), and longitudinal advance or withdrawal 125 of cryogenic fluid conduit 106. These motions are individually described, for example, in relation to FIGS. 2B-2C, 3, and 4.

Tip 111, in some embodiments, comprises a soft polymer; for example, Pebax®, polyurethane, polyethylene, and/or polyamide. The soft tip potentially reduces a risk of traumatic injury, and also, in some embodiments, provides sealing against the egress of coolant from longitudinally extended ablation element 102.

As an example: supply conduit 107, in some embodiments, has an inner diameter of about 0.2 mm, while an inner diameter of longitudinally extended ablation element 102 is about 1.1 mm.

The shaft of cryofluid conduit 106, in some embodiments, comprises a metal-reinforced polymer, for example a polymer reinforced with a braiding, helical wire, and/or laser cut tubing. Metal and plastic may be bonded, e.g., by glue. Optionally, a metal cap terminates a distal end of sheath 108. In the embodiment illustrated in FIGS. 1A-4, steering is implemented within sheath 108. Optionally, steering is implemented (additionally or alternatively) as part of cryofluid conduit 106 itself. This may change the available degrees of control.

Optionally (FIG. 1E), one or more thermocouples 171 are provided with cryoablation probe 101, for example, within and/or upon connector 104, tip 111, and/or longitudinally extended ablation element 102. The example of FIG. 1E shows thermocouple 171 mounted on a ring 174 (e.g., a ring of crimped-on metal), optionally embedded in welding 172. Wire 173 communicates signals from thermocouple 171 back through thermocouple conduit 175. Optionally, one or more electrophysiology electrodes are provided with cryoablation probe 101, for example, within, upon, and/or using connector 104, tip 111, and/or longitudinally extended ablation element 102. These may be mounted and connected, for example as indicated and described for thermocouple 171.

Reference is now made to FIG. 1B, which schematically represents a cryoablation device 100, including control elements, according to some embodiments of the present disclosure. The configuration of control elements is for purposes of providing an example for reference; control may be implemented by other combinations of elements, according to principles of engineering and construction known in the art. In the example shown, actuation is performed under manual control, e.g., controller housing 143 is sized to be held in the hands. In some embodiments, robotic actuation is provided for one or more of the controls.

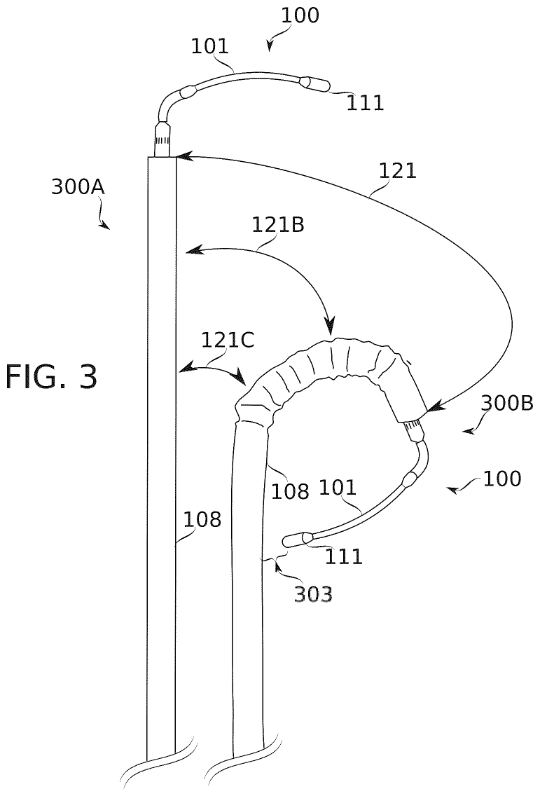

On the right side of the drawing of FIG. 1B, cryoprobe 101 is shown in a position extruded distally from sheath 108. Moving proximally, a section of sheath 108 between locations 146 and 147 is configured to bend under actuated control. The actuated control may be implemented, for example, by a wire 145. In some embodiments, wire 145 is fixedly secured at about location 147, while from location 146 and more proximally, sheath 108 is isolated from tension developed in wire 145, e.g., by passing wire 145 into a sheath 145A. Sliding slider 141 along slot 144—for example, in the directions indicated by arrows 161—increases or decreases tension on wire 145, resulting in bending of sheath 108 between locations 146 and 147, thereby implementing the induced curvature 121 of sheath 108. Bending is further described, for example, in relation to FIG. 3, herein.

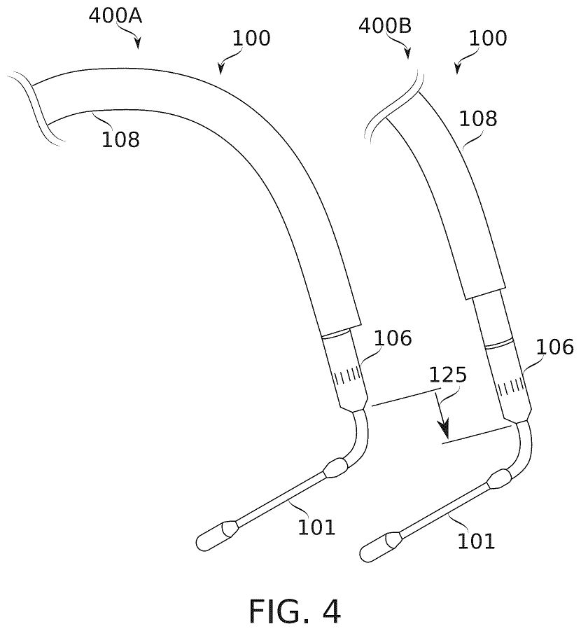

In some embodiments, knob 142 is rotatable relative to sheath 108 (double-arrowed curve 171), whereby the rotation 131 of cryogenic fluid conduit 106 and cryoablation probe 101 relative to sheath 108 is accomplished. Moving knob 142 longitudinally (double-arrowed curve 165) accomplishes longitudinal advance or withdrawal of cryogenic fluid conduit 106. These movements are further described, for example, in relation to FIGS. 2B-2C (for the rotation), and FIGS. 2A and 4 (for the longitudinal advance).

Cryogenic fluid is supplied to the cryoablation device through supply conduit 148. As drawn, supply conduit merges with a proximal end 106A of cryogenic fluid conduit on a proximal side of controller housing 143; alternatively, the merge may be, e.g., within controller housing 143, or on a distal side of controller housing 143.

Reference is now made to Figure/C, which schematically illustrates aspects of lumenal surface accessibility provided by the degrees of control freedom of a cryoablation device 100, according to some embodiments of the present disclosure.

In some methods of use, cryoablation catheter 100 is inserted to a body lumen. Lumenal surface 153 in FIG. 1C schematically represents the body lumen with a spherical shape; in some embodiments, lumenal surface 153 correspond to the lumenal surface of a heart chamber, for example, a left atrium. Tubular element 154 (representing sheath 108) passes through one side of lumenal surface 153. This corresponds, in some embodiments, to the entry of sheath 108 into a lumen, for example via an interatrial septum or a blood vessel. Some of the control motions indicated by arrows correspond to indicated already in relation to Figure/A: induced curvature 121 of sheath 108 (e.g., curvature induced to bend sheath-representing tubular element 154 away from axis 155); rotation 131 of cryogenic fluid conduit 106 (represented by inner tubular element 156) relative to sheath 108 (represented by tubular element 154); and longitudinal advance or withdrawal 131, also of cryogenic fluid conduit 106 relative to sheath 108.

Two other actuatable motions provide additional degrees of control freedom. Translation of tubular element 154 in the directions indicated by double arrow 133 corresponds to greater or lesser overall depth of insertion of the cryoablation device distal end into a body lumen, while rotation arrow 137 (again of tubular element 154) corresponds to rotation around axis 155 (rotation in either direction is optionally performed, and rotation can optionally be to any angle up to and optionally greater than 360°.

The different control motions allow various positionings of linear element 135 (representing cryoablation probe 101) upon lumenal surface 153. Circle 157 represents the sweep of the tip of linear element 135 resulting from rotation 131, with the other control settings remaining configured as shown. This circle can itself be swept around the interior of lumenal surface 153 by the rotation indicated by rotation arrow 137. This brings more area of lumenal surface 153 into reach, e.g., the whole of the surface defined between circles 151 and 152.

To reach more of lumenal surface 153, other combinations of control movement may be performed. If the bend angle of induced curvature 121 is made more obtuse, for example, then more distal regions of lumenal surface 153 may be accessible; or if made more acute, then more proximal regions of lumenal surface 153 may be accessible. To maintain surface contact, such a controlled motion is optionally accompanied by one or both of advancing longitudinally in accordance with the distal direction of double arrow 133, or advancing longitudinally in accordance with the distal direction of double arrow 125. Optionally, motion is distal for one of the longitudinal control options, and proximal for the other. Optionally, the longitudinal motions are used without changing the angle of induced curvature 121.

It should be noted in particular that there is available some configuration of movements which allow inner tubular element 156 to approach most positions across lumenal surface 153 perpendicularly. Regions which surround the position from which tubular element 154 enters lumenal surface 153 (e.g., approximately within the smaller zone circumscribed by circle 151) are an exception, sized according to limitations on the minimum radius of curvature through which is induced curvature 121 bends tubular element 151. At least in the idealized spherical case, a perpendicular approach will also maintain strain at curve 123 (corresponding to strain on angle-selecting connector 104) at about the same level as rotation 131 is changed.

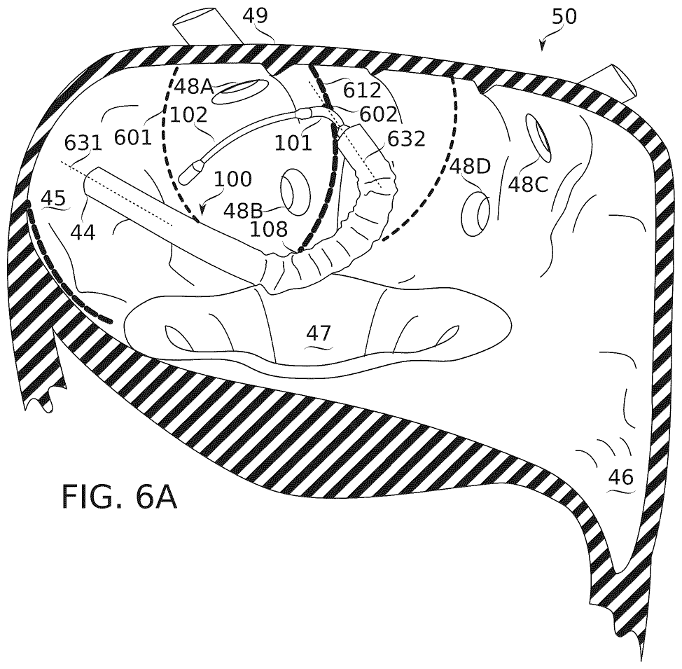

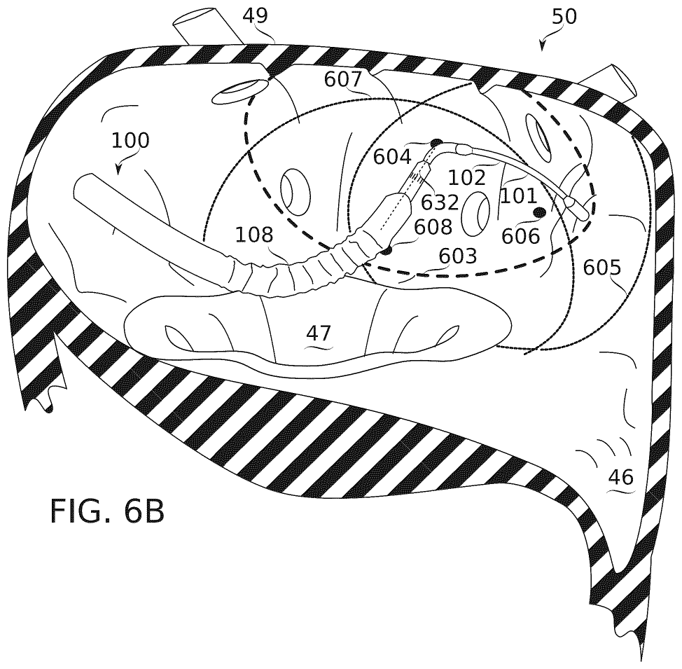

Optionally, a non-perpendicular approach is preferred, and the selection of controlled motions may alternatively be made to accomplish this, instead. Potential advantages of a non-particular approach are described, for example, in relation to FIGS. 6A-B and 7, herein.

### Cryocatheter Movements

Reference is now made to FIG. 2A, which schematically represents a sequence of configurations assumed by cryoprobe 101 during its advance from sheath 108, according to some embodiments of the present disclosure.

From right to left, the sequence shows:
In view 150A, cryoablation probe 101 has just begun to exit sheath 108, with tip 111 protruding. Linear ablation element 102 is still oriented along the longitudinal axis of the distal end of sheath 108.
In view 150B, cryoablation probe 101 has begun to tile obliquely, due to partial release of confinement allowing angle-selecting connector 104 to begin to relax into its preset configuration.
In view 150C, longitudinally extended ablation element is tilted further, after completely exiting sheath 108.
In view 150D, angle-selecting connector 104 has also exited the distal end of the catheter and assumed its relaxed (unconstrained) configuration. It may be noted that tip 111 is oriented at about a right angle to a distal longitudinal axis of sheath 108. Other portions of cryoablation probe 101 are at somewhat less than a right angle. For example, the proximal portion of longitudinally extended ablation element 102 is oriented at about a 75°-80° angle away from the longitudinal distal axis of sheath 108.

Angle-selecting connector 104, in some embodiments, is constructed from an inner portion 104, which may comprise a shape memory alloy such as nitinol, and optionally covered, by a short length of polymer tubing 104B. In some embodiments, flexibility of the inner portion 104A of angle-selecting connector 104 is enhanced by cutting away portions of a metal tube during manufacture (e.g., in a pattern of alternate-side cuts interleaved along the length of the connector). In some embodiments, angle-selecting connector 104 is formed as a metal coil. In the latter cases, the tubing 104B which covers the metal core of angle-selecting connector 104 also serves to maintain the sealed integrity of the cryofluid delivery conduits.

Reference is now made to FIGS. 2B-2C, which schematically represent rotational control of cryoprobe 101, according to some embodiments of the present disclosure. FIG. 2B shows an end-on view down the longitudinal extent of sheath 108, with cryoprobe 101 extending perpendicular to the direction of viewing. FIG. 2C shows a side view. Rotation of cryogenic fluid conduit 106 within sheath 108 is transmitted to cryoprobe 101, causing it to rotate through path 131. The extent of rotation may be any portion of a circle, up to and optionally beyond 360°. In some embodiments, longitudinally extended ablation element 102 is straight, for example, as depicted in FIGS. 2B-2C. Importing a slight preset curve to longitudinally extended ablation element 102 has potential advantages in conforming to the curved inner wall shape of a body cavity lumen, but even a straight tube may be deformable in operation, particularly if the tube comprises a shape memory alloy which has been cooled to at or near its critical temperature.

Reference is now made to FIG. 3, which schematically represents a sequence of configurations illustrating flexing control of catheter sheath 108 of a cryoablation device 100, according to some embodiments of the present disclosure. In view 300A, sheath 108 is straight, while in view 300B, sheath 108 is bent through an angle of about 155°. Optionally, the angle which can be achieved is at least 90°, and optionally the angle is up to 180° or more. Double-sided arrows 121, 121B, and 121C show approximate matches of the same portions of the curve as they are positioned straight and bent. Bending is optionally performed with our without cryoablation probe 101 extended; in the views shown, cryoablation probe 101 is extended.

In some embodiments, capability of bending through at least 135° is provided. In some embodiments, the bending angle can be increased to nearer or even above 180°. Even at 155°, it may be seen that cryoablation probe 101, in the orientation shown, is able to reach nearly all the way back to the shaft of sheath 108 when oriented to point at it. The minimal distance 303 between probe tip 111 and sheath 108 may be for example, less than 5 mm, less than 10 mm, or less than 20 mm.

Reference is now made to FIG. 4, which schematically represents a sequence of configurations illustrating translational control of cryoprobe 101 with respect to sheath 108, according to some embodiments of the present disclosure. Longitudinal extension (along arrow 125) of cryoablation catheter 100 is achieved by translating cryogenic fluid conduit 106 longitudinally relative to catheter sheath 108 (e.g., while catheter sheath 108 remains stationary). This adjustment provide a ready means for advancing cryoablation 101 as far as necessary to bring it into contact with a tissue wall. View 400B shows a relatively extended configuration, compared to view 400A.

### Methods of Operating Cryocatheters

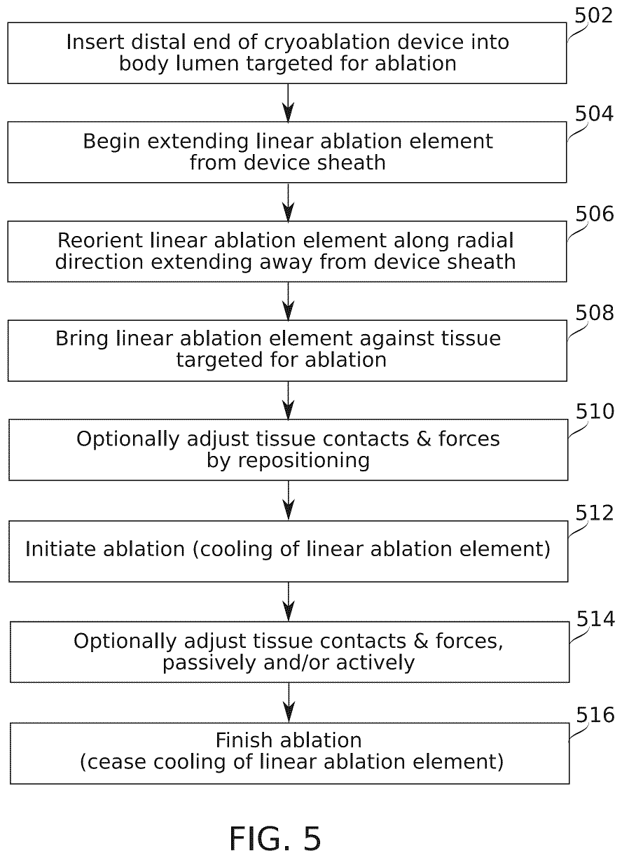

Reference is now made to FIG. 5, which is a flowchart schematically representing a method of operation of cryoablation device 100, according to some embodiments of the present disclosure.

At block 502, in some embodiments, a distal aperture of a sheath 108 of a cryoablation catheter 100 is inserted to a body lumen.

At block 504, in some embodiments, a longitudinally extended ablation element 102 of the cryoablation catheter 100 begins to be extended longitudinally along a longitudinal axis defined by an orientation of the distal end of the sheath 108.

At block 506, in some embodiments, and as longitudinally extended ablation element 102 extends, it reorients so that it extends along a new axis extending more radially from the longitudinal axis of the sheath 108. In some embodiments, the longitudinally extended ablation element 102, measured from a distal end to a proximal end thereof, extends at an angle which is oblique by an angular measure of less than 20° to an axis making a right angle with the longitudinal axis of the sheath 108.

In some embodiments, the proximal end of the longitudinally extended ablation element 102 remains within about 2 cm or within about 1 cm of the longitudinal axis of the sheath during the reorientation. In some embodiments, a connector 104 which is attached to the proximal end of the longitudinally extended ablation element 102 bends to induce the reorientation. In some embodiments, connector 104 is set to bend to a predetermined angle upon the removal of constraints that otherwise tend to straighten it, and this predetermined angle selects the re-oriented angle of the longitudinally extended ablation element 102. The predetermined angle may vary slightly from repetition to repetition, e.g., with 95% of trials (e.g., 20 trials) remaining within a range of about ±5°. Variation may be due, e.g., to hysteresis in the elasticity of the materials of the device, and/or friction. The reproducibility test suggested should be performed with external factors controlled; e.g., the orientation of the device with respect to the pull of gravity.

It should further be noted that since connector 104 is flexible, forces exerted on it or on longitudinally extended ablation element 102 could deflect longitudinally extended ablation element itself away from the re-oriented angle; the re-oriented angle referred to is an angle which is taken up in the absence of such forces due, e.g., to interference from tissue of a body lumen in which the cryoablation device 100 is being operated. In some embodiments, connector 104, when freed to bend, bends through a radius of curvature less than about 2 cm, or less than about 1 cm where it bends to induce the reorientation of longitudinally extended ablation element 102.

Elongated ablation element 102 itself is configured, in some embodiments, as a tubular structure (e.g., with a diameter of 2 mm or less, 1.8 mm or less, 1.5 mm or less, or 1 mm or less), adapted for the circulation therethrough of a cryofluid having, e.g., a temperature of −40° C. or less. In some embodiments, the distance between the aforementioned distal end and proximal end of the longitudinally extended ablation element 102 is at least 10 mm, 12 mm, 13 mm, 15 mm, 2 cm, or 3 cm In some embodiments, longitudinally extended ablation element 102 curves along this distance, with lines tangent to this curvature along its length differing from each other in angle by not more than 15°, 30°, or 45°. In some embodiments, longitudinally extended ablation element 102 is straight.

At block 508, in some embodiments, longitudinally extended ablation element 102 is further reoriented and/or advanced by the controlled manipulation of degrees of freedom of cryoablation device 100, to bring it into contact with tissue of the body lumen which is targeted for ablation. Control comprises movement of an actuator device, movement of the whole device, and/or movements of parts of the device relative to each other, for example as described in relation to FIGS. 1A-4.

In some embodiments, the re-orientation of block 506 orients a convex side of the longitudinal curvature to face away from the direction of sheath 108, and a concave side of the longitudinal curvature to face in a direction facing closer to the direction of sheath 108. The convex side of the longitudinal curvature is oriented so that—e.g., as part of the operations of block 508—advancing the longitudinally extended ablation element 102 in a distal direction brings it into contact with the walls of the body lumen. At this location, the convex side may act as an ablation surface, when cryoablation probe 101 (of which longitudinally extended ablation element 102 is a part) is activated by the flow of coolant.

Optionally, at block 510, in some embodiments, contact of longitudinally extended ablation element 102 with tissue of the body lumen is adjusted by further manipulation of the controlled degrees of freedom of cryoablation device 100. In some embodiments, the adjustments alter the obliquity of the longitudinal axis of the sheath 108 relative to a body lumen tissue surface that the longitudinal axis intersection, while longitudinally extended ablation element 102 remains in contact with the tissue contacted in block 108. In some embodiments, the altered obliquity results in a change in the curvature of connector 104. In some embodiments, the altered obliquity redistributes contact force along an ablation surface of longitudinally extended ablation element 102. In some embodiments, contact force is redistributed toward the tip of longitudinally extended ablation element 102, e.g., with 80% or more of contact force focused in the distal-most 50% of longitudinally extended ablation element and/or a tip 111 thereof.

At block 512, in some embodiments, longitudinally extended ablation element 102 is cooled, e.g., cooled by circulation of a coolant therethrough.

Optionally, at block 514, in some embodiments, force along ablation element 102 is redistributed again. This may have the potential advantage of helping to sure that ablation occurs along the whole extent of longitudinally extended ablation element 102. In some embodiments, longitudinally extended ablation element 102 and/or connector 104 comprises a material which softens as a result of the cooling (e.g., a superelastic and/or shape-memory metal allow such as nitinol). With exerted contact forces remaining stable, the softening may nevertheless passively redistribute them so that, e.g., more proximal regions of the longitudinally extended ablation element 102 are brought into increasing contact (contact with stronger force) with tissue. Optionally, active redistribution of force is performed, e.g., by reorienting the obliquity of the device, for example as described in relation to block 510.

Freezing of portions of longitudinally extended ablation element 102 to tissue may help to maintained continued tissue contact during cryoablation. The overall length of scar is optionally the whole length of longitudinally extended ablation element 102. Optionally, a smaller length is selected, e.g., by freezing a distal portion of longitudinally extended ablation element 102 in strongest contact with tissue, and then pulling the device partially away before contact from a more proximal portion of longitudinally extended ablation element 102 induced ablation. Again, low-temperature softening of the material of elongated ablation probe 102 and/or connector 104 may assist in achieving this control.

There is optionally further control of the development of chilled temperatures along the longitudinal extent of longitudinally extended ablation element 102 by a predetermined and/or actuated selection of the location of internal apertures at which coolant depressurizes within cryoablation probe 101; e.g., nearer to its distal tip, or nearer to its proximal side.

At block 516, in some embodiments, the circulation of coolant is stopped, ablation ceases, and the flowchart of FIG. 5 ends.

### Cryocatheters Operation In Situ

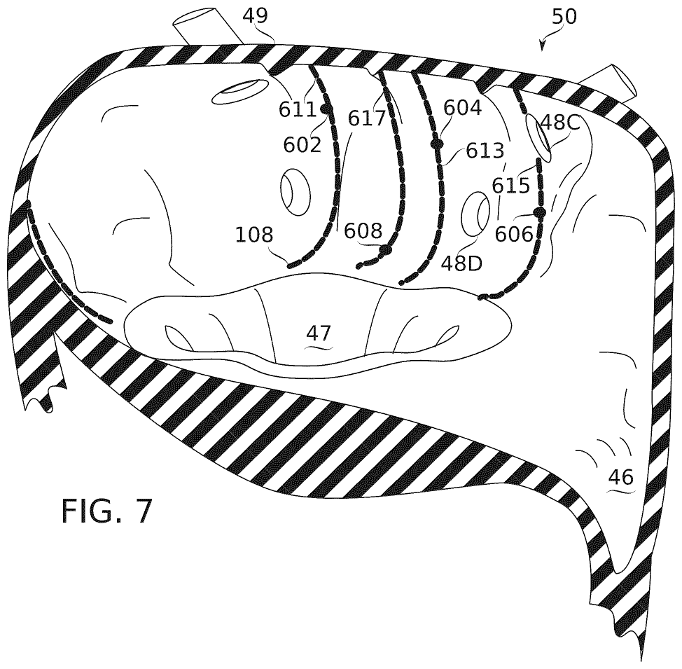

Reference is now made to FIGS. 6A-7, which schematically illustrate positioning of cryoablation device 100 within a left atrium 49 of a heart 50, according to some embodiments of the present disclosure. Other anatomical landmarks of the left atrium represented include four ostia 48A-48D of the pulmonary veins, mitral valve 47, and left atrial appendage 46.

In FIG. 6A, cryoablation device 100 is inserted to the left atrium 49 across the interatrial septal wall 45, for example via fossa 44.

Linear ablation element 102 is placed against a proximal wall region, between pulmonary vein ostium 48A, and pulmonary vein ostium 48B. In the example shown, this involves bending sheath 108 through all or nearly all of its range, and then orienting cryoablation probe 101 by rotation to point back toward the sheath 108. Further selection of the contact position is adjusted by rotating the whole device around axis 631. Linear ablation element 102 can be snugly fitted to tissue by pulling the whole device proximally. Resistance to pulling indicating a good fit may be felt, and/or viewed angiographically, e.g., evidenced by deflection of angle-selecting connector 104 in response to movement of the device and/or of the heart atrium itself as it beats.

Indicated for purposes of reference is region 602, which is located where longitudinal axis 632 extending from the distal tip of sheath 108 intersects tissue. This region is also near the region of proximal contact of probe 101 to tissue, so it serves as a convenient indicator of which areas of tissue can be reached by probe 101. Heavy dashed line 612 shows the path along which the intersection point can be moved as the whole sheath 108 is rotated around axis 631. Lighter dotted line 601 shows, as a rough circle, the outer circumference of the sweep range of cryoablation 101 if it is rotated around axis 632. It may be readily understood that just operating these two degrees of control freedom together, allows a wide band of tissue to be reached for ablation, which extends by the radius of circle 601 to either side of dashed line 612.

In FIG. 6B, sheath 108 is unbent by about 60°, and also adjusted somewhat in its penetration depth into the right atrium. Now a different tissue region 604 comes into intersection with axis 632, surrounded by a different circle of cryoablation probe access 603. Additional examples of axis intersection/circle of access pairs include region 606 matched to path 605, and region 608 matched to path 607.

FIG. 7 shows pathways 611, 617, 613, and 615; along which regions 602, 608, 604, and 606 respectively are positioned. Each path is defined rotation of a cryoablation device 100 around axis 631 with a different arrangement of other position controls. It may be noted that different base positions allow different access to similar tissue regions from different angles. For example, the region between pulmonary vein ostia 48C and 48D may be bisected by stretching across it from either path 613 or path 606 at any of a range of orientations of the cryoablation probe.

Examples of locations in the left atrium along which ablation lines may be performed include:
Posterior wall isolations—lines isolate the ostia of the pulmonary veins 48A-48D; individually (e.g., and from each other) and/or as pairs or larger groupings.
An ablation extending along the roof of the left atrium, e.g., from the left side to the right side of any of FIGS. 6A-7, and along the top of the lumenal surface.
A “left isthmus” line, crossing the annulus of mitral valve 47.
LAA isolations, i.e., lines ablated around the opening in the left atrial appendage 46.

Optionally or alternatively, one or more ablation isolations are performed in the right atrium; for example, along the cavotricuspid isthmus line (e.g., between the connection of the vena cava with the right atrium, downward to the annulus of the tricuspid).

It has been mentioned in relation to FIG. 1C that more perpendicular or less perpendicular angles of approach are optionally selected. A more perpendicular approach potentially gives some measure of enhancement of positioning control, since there is then less lateral force which may tend to let the cryoablation probe 101 slip sideways.

However, the non-perpendicular angles also have some potential advantages for control, which can be useful, e.g., where surfaces and curvatures are irregular. In particular, changing the angle of approach can be used to modify the magnitude and/or distribution of forces which a longitudinally extended ablation element 102 exerts upon lumenal surface, e.g., of left atrium 49. For example, it may be advantageous to position angle-selecting connector 104 so that its bend is forced to become more acute than its natural shape (e.g., bent partially backward along sheath 108). This may allow contact force to be focused preferentially, e.g., nearer to a distal end of longitudinally extended ablation element 102. Contact force and its distribution potentially influence the length, positioning, and/or quality of cryoablation lesioning accomplished.

Optionally, control position adjustments are performed part way through creation of a cryoablation lesion. For example, an acute bend through angle-selecting connector 104 may be established at an approach angle which favors increased pressure at a distal tip of longitudinally extended ablation element 102. This may cause regions near the distal tip 111 to stick by freezing to tissue at a period when freezing is beginning. After this, changes in angulation and/or force can be made which adjust force to favor contact with more proximal regions of the ablation surface of longitudinally extended ablation element 102. Even without active adjustment, a superelastic metal used to construct longitudinally extended ablation element 102 is optionally selected so that cryoablation cools it to near or below its critical temperature. At that temperature, softening of ablation element 102 may itself redistribute force, so that more proximal regions of the ablation element 102 are also brought into increased contact with tissue.

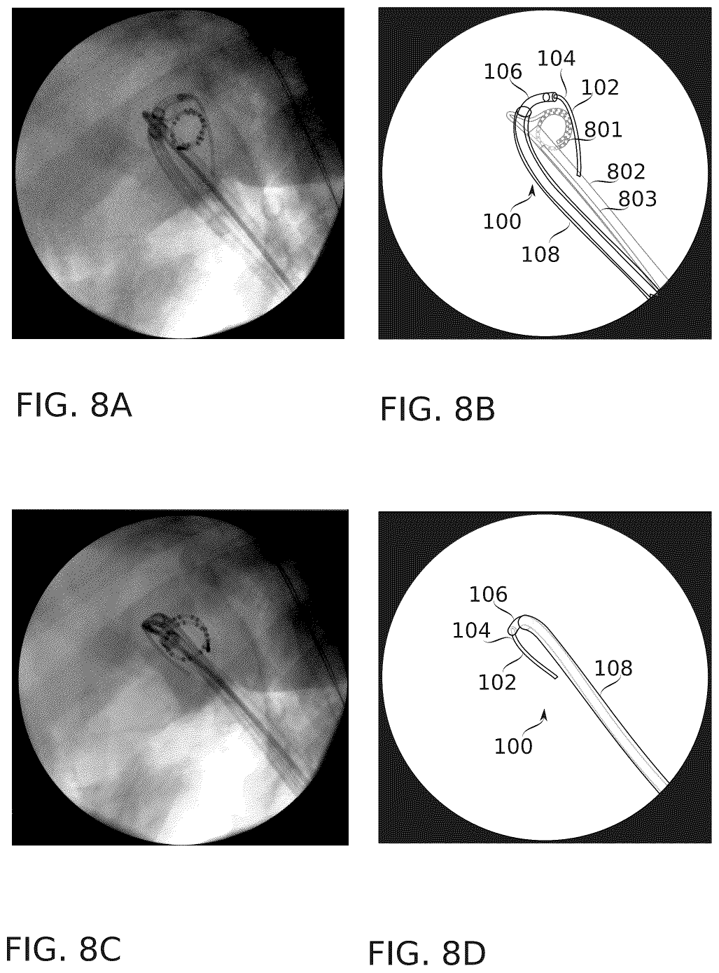

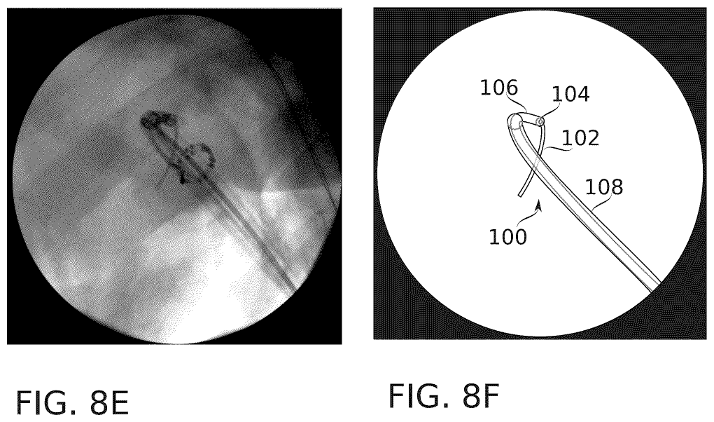

Reference is now made to FIGS. 8A-8F, which schematically and with angiographic images represents positioning of cryoablation device 100 within a left atrium 49, according to some embodiments of the present disclosure. The figures also illustrate a mode of using cryoablation device 100, in which a multielectrode catheter 801 (formed in a looped, spiral, and/or circular shape, for example as shown in the images and in FIG. 8B) is used to sense locations where prior ablation gaps can be detected, allowing the longitudinally extended ablation element 102 to be positioned alongside and operated to create a new lesion, closing the gap.

FIGS. 8A, 8C, and 8E reproduce angiographic images of the operation of cryoablation device 100 within a heart chamber of an animal. FIGS. 8B, 8D, and 8F are drawings which correspond respectively to the three angiographic images. The drawing may assist the understanding of features described in relation to configurations shown in the angiographic images.

FIG. 8B draws the relative positions of both multielectrode catheter 801 and cryoablation device 100. Both devices have been inserted to the same heart chamber via trans septal access (from the lower right corner of the image). A proximal portion 803 of the electrode catheter extends within sheath 802, terminating in the coiled arrangement of electrodes (assuming a ring shape) which is the electrically sensing portion of the multielectrode catheter 801. In the situation shown, the coiled region of multielectrode catheter 801 is positioned to surround a region which may be one from which ectopic electrical impulses are escaping; for example, the loops of multi electrode array 801 may be inserted into the ostium of a pulmonary vein from which impulses are arising that are to be blocked. For example as may occur after an incompletely isolated ablation procedure. According to relative magnitude and/or timing of electrical signal sensing, it can be determined which electrodes are closest to the gap region. From this information, it can be decided how to position the longitudinally extended ablation element 102 so that the gap can be closed by a further ablation.

Optionally, electrodes attached to cryoablation device 100 itself (e.g., as part of cryoablation probe 101) are used to detect and/or confirm gaps in previous ablations, and/or identify regions where electrical impulse timing is indicative of regions in need of electrical isolation.

The three figure pairs show how manipulation of the control degrees of freedom of the device allows ready placement of the longitudinally extended ablation element along of different sides of coiled electrode array. In the drawn member of the second two figure pairs, only cryoablation device 100 has been reproduced, so that its positioning can be more clearly discerned. The positioning of the coil of the multielectrode catheter 801 remains approximately the same throughout.

In FIGS. 8A-8B, longitudinally extended ablation element 102 is placed to extend along a right-hand side of the coil of electrode catheter 801. General positions of other controllable and/or flexible elements 104, 106, and 108 are also shown.

In FIGS. 8C-8D, longitudinally extended ablation element appears on the opposite side of the same coil. There is a significant change in the foreshortening of the curvature of sheath 108 in this image. Finally, FIG. 8E-8F show the longitudinally extended ablation element 102 crossed under sheath 108 to another orientation and positioned against a third side of the coil of multielectrode catheter 801.

It may be noted that in each of FIGS. 8A, 8C, and 8E, the longitudinally extended ablation element is placed about parallel to a tangent of the adjacent ring of coils. This positioning potentially assists in generating new lesion lines in a substantially ring-shaped (polygonal) configuration, and/or in closing off gaps in substantially ring shaped previously created lesion lines.

### Specialized-Shape Cryocatheters

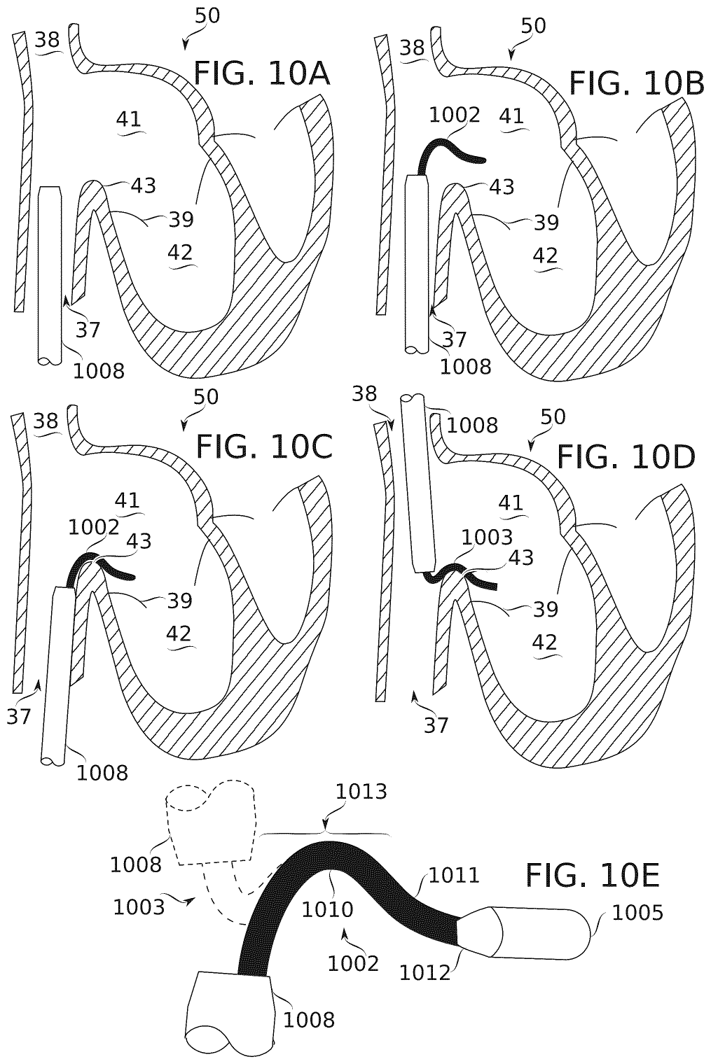

Reference is now made to FIG. 10A-10D, which schematically illustrate use of longitudinally extended ablation elements 1002, 1003, shaped to conform to the shape of a targeted ablation region, while avoiding tissue of adjacent non-targeted tissue, according to some embodiments of the present disclosure. Reference is also made to Figure JOE, which schematically illustrates expanded views of longitudinally extended ablation elements 1002, 1003 according to some embodiments of the present disclosure.

In FIG. 10A, catheter 1008 is being advanced into right atrium 41 via inferior vena cava 37 of a heart 50. An example of a specially-shaped region targeted for ablation is indicated as cavo-tricuspid isthmus 43, which, as the name suggests, is a ridge of tissue extending between the tricuspid valve 39 (which itself lies between the right ventricle 42 and the right atrium 41), and the inferior vena cava 37. This area may be involved in fibrillation pathologies which arise from the establishment of an abnormal ring-like transmission pathway of heart electrical impulses. Ablating at the cavo-tricuspid isthmus 43 interrupts the transmission ring.

In Figure JOB, longitudinally extended ablation probe 1002, shaped as a hook with a refused distal end is exposed from within catheter 1008, and oriented to extend into the right atrium 41, and above the cavo-tricuspid isthmus. In FIG. 10C, the hook is placed upon cavo-tricuspid isthmus 43 by pulling it proximally. It fits to the cavo-tricuspid isthmus 43 along its main body 1013 including curvature 1010 (Figure JOE), while more proximally it changes the direction of its curve (curvature 1011), which may tend to keep it away from the valve leaflets; or if it does contact tissue distally, the contact doesn't prevent the more proximal curvature from engaging in contact too. The more proximal curvature may itself contribute to the ablation surface of ablation element 1002, or it may be an extension which helps to keep tip 1005 (which, in some embodiments, is somewhat enlarged larger in diameter optionally via expanding region 1012, e.g., 50% or more enlarged) away from a blocking and/or damaging position.

FIG. 1D shows a variant longitudinally extended ablation element 1003 which can be brought in from the superior vena cava 38. Here, there is a third (and still more proximal) curvature 1003 provided, since the initial direction of curvature is inverted from its approach. In Figure JOE, this curvature is shown in dotted lines, indicating an alternative to the proximal continuation from curve 1010 toward the solid-line drawing of catheter 1008.

It should be noted that catheter 1008 is optionally provided without steering, since its natural line of approach already places it in position.

### General

As used herein with reference to quantity or value, the term “about” means “within ±10% of”.

The terms “comprises”, “comprising”, “includes”, “including”, “having” and their conjugates mean: “including but not limited to”.

The term “consisting of” means: “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

The words “example” and “exemplary” are used herein to mean “serving as an example, instance or illustration”. Any embodiment described as an “example” or “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the present disclosure may include a plurality of “optional” features except insofar as such features conflict.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

Throughout this application, embodiments may be presented with reference to a range format. It should be understood that the description in range format is merely for convenience and brevity and should not be construed as an inflexible limitation on the scope of descriptions of the present disclosure. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Although descriptions of the present disclosure are provided in conjunction with specific embodiments, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

It is appreciated that certain features which are, for clarity, described in the present disclosure in the context of separate embodiments, may also be provided in combination in a single embodiment. Conversely, various features, which are, for brevity, described in the context of a single embodiment, may also be provided separately or in any suitable subcombination or as suitable in any other described embodiment of the present disclosure. Certain features described in the context of various embodiments are not to be considered essential features of those embodiments, unless the embodiment is inoperative without those elements.

1. A cryoablation device configured to be inserted to a heart chamber via a transcutaneous, transvascular route, and comprising:
a sheath, the sheath being actuatable to bend and thereby rotate a longitudinal axis of a distal end of the sheath to a selected angle within a range of angles between a straightened sheath angle, and at least 90° away from that angle;
within the sheath, a cryogenic fluid conduit, actuatable to translate linearly along the longitudinal axis with respect to the sheath;
a longitudinally extended ablation element:
attached to a distal end of the cryogenic fluid conduit,
sized to fit within the distal end of the sheath, and
configured to receive coolant from the cryogenic fluid conduit to cool down to generate a longitudinally extended cryoablation surface along a longitudinal extent of the longitudinally extended ablation element; and
an angle-selecting connector between the longitudinally extended ablation element and the fluid supply conduit, the angle-selecting connector bending to orient the longitudinally extended ablation surface to within about 20° of perpendicular to the longitudinal axis when the longitudinally extended element is outside the sheath.

2. The cryoablation device of claim 1, wherein the longitudinally extended ablation surface extends at least 13 mm along the longitudinally extended ablation element, and the longitudinally extended element curves less than 45° along a curvature following the at least 13 mm extent of the longitudinally extended ablation surface.

3. (canceled)

4. The cryoablation device of claim 1, wherein the longitudinally extended ablation element comprises a proximal side and a distal side, the proximal side is attached to the angled connection, and the distal side comprises a terminus with a tip.

5. (canceled)

6. The cryoablation device of claim 1, wherein the sheath holds the angle-selecting connector straightened while the longitudinally extended element is housed within the sheath, and the angle-selecting connector is configured to bend as the longitudinally extended element emerges from the sheath.

7. The cryoablation device of claim 1, wherein the angle-selecting connector comprises a metal piece with slits along its sides, and enclosed in flexible polymer tubing.

8. The cryoablation device of claim 7, wherein the slits along the sides comprise gaps between coils of the angle-selecting connector.

9. The cryoablation device of claim 7, wherein the slits along the sides comprise perforations positioned in opposite sets, to allow the metal piece to form a bend by contracting the perforations on an inner side of the bend, and expanding the perforations on an outer side of the bend.

10. (canceled)

11. The cryoablation device of claim 1, wherein the angle-selecting connector is short enough to allow the sheath distal end to remain within about 1 cm of the cryoablation surface of the longitudinally extended ablation element when deployed to contact and ablate tissue.

12. The cryoablation device of claim 1, wherein the angle-selecting connector is short enough that a proximal end of the longitudinally extended ablation element remains within about 1 cm of longitudinal axis when both it and the angle-selecting connector are emerged from within the sheath.

13. The cryoablation device of claim 1, wherein the angle-selecting connector bends through a radius of curvature in a range of about 3-10 mm.

14. The cryoablation device of claim 1, wherein, once the angle-selecting connector and the longitudinally extended ablation element are emerged from the sheath, the cryogenic fluid conduit is configured to allow further actuated advance along the longitudinal axis, while the sheath distal end remains in place, and wherein the cryogenic fluid conduit is configured to rotate within the sheath, thereby selecting an angular position of the longitudinally extended ablation element around the longitudinal axis when emerged from the sheath.

15. (canceled)

16. The cryoablation device of claim 1, wherein the distal end of the sheath is bendable to assume at least 135° of curvature, while the longitudinally extended ablation element is emerged from the sheath.

17-18. (canceled)

19. The cryoablation device of claim 1, wherein the longitudinally extended ablation element comprises a superelastic alloy which softens at the cryoablation temperature to allow redistribution of contact forces between the distal end of the longitudinally extended ablation element and the proximal side of the longitudinally extended ablation element.

20. The cryoablation device of claim 1, comprising at least one thermocouple device positioned between the cryogenic fluid conduit and a distal tip attached to the distal end of the longitudinally extended ablation element.

21. The cryoablation device of claim 1, comprising at least one electrode attached to the longitudinally extended ablation element, and configured to sense electrophysiological signals from tissue while the ablation surface is pressed into contact with tissue targeted for ablation.

22. (canceled)

23. The cryoablation device of claim 1, comprising a handle with an actuation control for linear translation of the cryogenic fluid conduit relative to the sheath wherein the handle comprises an actuation control for rotating the cryogenic fluid conduit relative to the sheath, and an actuation control for bending the sheath.

24-25. (canceled)

26. The cryoablation device of claim 1, wherein the longitudinally extended ablation element comprises:
a first curvature distal to curvature of the angle-selecting connector; and
a second curvature distal to the first curvature, and in an opposite direction;
wherein the first curvature is oriented to fit against an interior surface portion of a body tissue lumen, and the second curvature is oriented to elevate a tip of the longitudinally extended ablation element away from tissue adjacent to the interior surface portion.

27. (canceled)

28. A method of operating a cryoablation device comprising:
inserting a sheath of the device through an entry aperture in a tissue wall;
bending a portion of the sheath inserted past the tissue wall to an angle of more than 90°;
linearly translating a cryofluid conduit along a longitudinal axis of a distal portion of the sheath, causing a longitudinally extended ablation element to emerge from the sheath and rotate to assume an orientation within about 20° of a plane perpendicular to the longitudinal axis;
rotate the longitudinally extended ablation element around the longitudinal axis by rotation of the cryofluid conduit; and
further linearly translating the cryofluid conduit to bring a cryoablation surface of the longitudinally extended ablation element into contact with tissue of the tissue wall, within 2 cm of the entry aperture.

29. The method of claim 28, comprising partially withdrawing the inserted portion of the sheath to bring the cryoablation surface into contact with tissue of the tissue wall.

30. The method of claim 28, comprising bending the portion of the sheath through an angle of more than 135°.

31-50. (canceled)
